(19) United States
(12) Patent Application Publication
(10) Pub. No.: US 2003/0091583 A1
(43) Pub. Date: May 15, 2003

(54) Use of mia in immunotheraphy

(76) Inventors: Nelissen Robert Louis Hubert, Oss (NL); Verheijden Gijsbertus Franciscus Maria, Oss (NL)

Correspondence Address: William M Blackstone, Intervet Inc Patent Department, 405 State Street, PO Box 318, Millsboro, DE, 19966, US

(21) Appl. No.: 10/239,251

(22) PCT Filed: Mar. 15, 2001
(86) PCT No.: PCT/EP01/02991

(30) Foreign Application Priority Data
Mar. 23, 2000 (NL) 00201063.5

(51) Int. Cl. A61K 39/00

(52) U.S. Cl. 424/185.1

(57) ABSTRACT

The invention relates to the use of MIA to prevent inflammatory diseases and in particular their use in treatment of chronic destruction of articular cartilage. More specifically, MIA can be used to induce specific T-cell tolerance to the MIA antigen in patients suffering from rheumatoid arthritis.

The current invention relates to the use of the MIA protein or specific derivatives thereof as immune modulatory agents for the treatment of autoimmune diseases, and more specifically rheumatoid artritis.

The primary functional role of the immune system is to protect the individual against invading pathogens bearing foreign that is non-self, antigens. In order to fulfil this function in a safe and effective manner, a mechanism is required to discriminate between foreign antigens and autoantigens derived from the individuals own body. Failure of this process of self-non-self discrimination, that is loss of immune tolerance to self-antigens, may lead to immune reactivity to autoantigens resulting in autoimmune disease, involving tissue damage and loss of organ function.

Autoimmune diseases are a major problem in human health care. Some autoimmune diseases may be the result of an immunological process directed at one antigen or antigenic complex whereas in others the autoimmune reaction may involve many types of antigens that may be present in multiple organs. Several lines of evidence have indicated that the immune system is involved in the pathology of autoimmune diseases. First, the chances of individuals to develop an autoimmune disease are closely linked to their genetic backgrounds: genes encoding major histocompatibility complex (MHC) class II molecules that present (auto)antigens to responding T cells which recognise MHC-peptide complexes show a strong genetic linkage to disease susceptibility. Second, cells of the immune system such as monocyte/macrophages and T cells infiltrate target organs. Third, T cells of patients with autoimmune diseases proliferate in vitro in response to potentially involved autoantigens. Fourth, studies in animal models of autoimmunity have unequivocally demonstrated that cells of the immune system such as monocyte/macrophages and T cells are involved in induction and expression of disease activity.

A disease as rheumatoid arthritis (RA) can illustrate the immunopathology that may occur in case of an autoimmune disease. RA presents itself as a chronic multisystem disease in which the common clinical manifestation is the persistent inflammatory synovitis accompanied by proliferation of synovial cells, pannus formation, cartilage degradation and bone erosion, and ultimately joint deformity resulting in loss of function.

Existing therapies for the treatment of autoimmune disorders, such as RA, in which the immune system generates an unwanted and undesirable inflammatory response, are inadequate. Treatment has focused on relief of symptoms of autoimmune disease rather than on its cause. Most drugs used in the treatment of autoimmune diseases, e.g. steroids and non-steroidal anti-inflammatory compounds, are non-specific and have significant toxic side effects. This is especially problematic since autoimmune diseases are chronic conditions, which require the prolonged administration of drugs.

Antigen-specific, non-toxic immunomodulation therapy provides a very attractive alternative for the non-specific immunosuppression. This antigen-specific therapy involves the treatment of patients with the target (auto)antigen or with synthetic T cell-reactive peptides derived from the (auto)antigen. These synthetic peptides correspond to T cell epitopes of the (auto)antigen and can be used to induce specific T cell tolerance both to themselves and to the (auto)antigen. The controlled administration of the target (auto)antigen can be very effective in desensitisation of the immune system. Desensitisation or immune tolerance of the immune system is based on the long-observed phenomenon that animals which have been fed or have inhaled an antigen or epitope are less capable of developing a systemic immune response towards said antigen or epitope when said antigen or epitope is introduced via a systemic route.

It has now been found that the protein called melanoma inhibiting activity (MIA) can be used in modulating the immune system.

Secreted protein fractions of melanoma cells containing MIA have first been reported by Bogdahn et al (1989, Cancer Res. 49:5358-5363). Purified MIA inhibited melanoma cell proliferation by prolongation of the S-phase and arrest in the G2 compartment. Upon purification of the MIA protein and partial amino acid sequencing, the cDNA encoding MIA protein was identified using degenerated oligonucleotides (Blesch et al, 1994, Cancer Res. 54:5695-5701). The protein appeared to be translated as a 131 amino acid precursor, that is processed into the mature 107 aa MIA by cleavage of a putative secretion signal peptide. No homology to any known protein was found. Isolation of the mouse counterpart cDNA of MIA revealed a high evolutionary conservation, since it encoded for a protein with 88% amino acid identity to the human protein. Human melanoma cell lines were shown to secrete the 11 kD MIA protein into the culture medium. Purified MIA protein that was secreted by melanoma cell line HTZ-19 or that was produced in E. coli appeared to act as potent cell growth inhibitor for malignant melanoma cells and some neuroectodermal tumours. Based on the growth-regulatory characteristic, MIA was suggested to be attractive as an anti-tumour therapeutical substance. Purified MIA containing a C-terminal histidine tag next to Cys-130 was reported to be totally inactive in growth inhibition assays.

Van Groningen et al. (1995, Cancer Res. 55:6237-6243) showed that MIA gene expression was detected in non-metastasising melanoma cell lines and in melanoma metastasis lesions, but not in highly metastasising cell lines and pretumor stages. The structure of the human MIA gene was reported by Bosserhoff et al. (1996, Anticancer Res. 19:2691-2693) and its promoter gave high levels of gene activation specifically in human and murine melanoma cells and its activity could be enhanced by treatment with phorbol esters.

On protein level Bosserhoff et al. (1997, Developm. Dynamics 208:516-525; 1999, Anticancer Res. 19:2691-2693) concluded that enhanced serum levels of MIA in patients with malignant melanoma are closely associated with late stages of the disease.

Studying the effect of retinoic acid on gene expression in bovine articular cartilage, Dietz and Sandell (1996, J. Biol. Chem. 271:3311-3316) cloned the cDNA of the inhibited CD-RAP gene (cartilage-derived retinoic acid-sensitive protein). CD-RAP was concluded to be the bovine counterpart of the human MIA.

From in situ hybridisation and immunolocalization on mouse and rat tissues it was concluded that the normal expression of CD-RAP is limited to cartilage. The expression of the CD-RAP/MIA gene seemed to be associated with chondrogenesis.

Recently, enhanced serum levels of MIA were also reported in patients with rheumatic diseases, e.g. rheumatoid arthritis, associated with joint destruction (Müller-Ladner et al, 1999, Rheumatol. 38:148-154) and in marathon-runners following their effort (Neidhart et al, 1999, Abstract 1412, American Coll. Rheumatol.—63^{rd} Annual Sci. Meeting). Thus, there seems to exist a diagnostic relationship between the presence of enhanced serum levels of MIA and damage to joint tissue.

The main problem in (auto)immune diseases (such as e.g. RA) is that the precise targets or antigens that the immune system is adversely reacting to are largely unknown, implicating that modulating a disease entity in an antigen-specific fashion may not be possible.

It would be an important advantage, however, if an antigen-driven, non-toxic form of immunomodulation therapy could be utilised without knowledge of the antigen(s) that are involved as a target in the (auto)immune response. Such an antigen-driven therapy would involve the generation of antigen-specific modulator cells with the use of an antigen that is expected to be released or produced during the autoimmune process. Such an antigen would become available during inflammation or tissue destruction. In case of an autoimmune disease, the locally produced autoantigen should then activate or reactivate modulator cells induced with such an antigen.

To effectively use tolerance induction therapy to treat T cell mediated cartilage destruction, there is a great need to identify T cell-reactive (poly)peptides which can desensitise patients against the autoantigen that is activating the T cells responsible for the inflammatory process.

It is an object of the invention to provide a (poly)peptide which is capable of inducing systemic immune tolerance, more in particular specific T cell tolerance, preferably to the responsible cartilage antigen in patients suffering from T cell-mediated cartilage destruction. It has now been found that MIA fulfils the above mentioned requirements and can be used as an effective toleragen.

In the present invention under induction of systemic immune tolerance is to be understood the stimulation-of antigen specific lymphocytes by antigen presenting cells (APC) in such a way that the lymphocytes acquire a state in which they produce anti-inflammatory cytokines. Anti-inflammatory cytokines may for example be IL-4, IL-10, and/or TGF-β. Lymphocytes brought to tolerance by APC are able to impose their anti-inflammatory state to other sites of the body, e.g. sites of ongoing inflammation.

The immune system protects individuals against foreign antigens and responds to exposure to a foreign antigen by activating specific cells such as T- and B-lymphocytes and producing soluble factors like interleukins, antibodies and complement factors. The antigen to which the immune system responds is degraded by the antigen presenting cells (APCs) and a fragment of the antigen is expressed on the cell surface associated with a major histocompatibility complex (MHC) class II glycoprotein. The MHC-glycoprotein-antigen-fragment complex is presented to a T cell, which by virtue of its T cell receptor recognises the antigen fragment conjointly with the MHC class II protein to which it is bound. The T cell becomes activated, i.e. proliferates and/or produces interleukins, resulting in the expansion of the activated lymphocytes directed to the antigen under attack (Grey et al., Sci. Am., 261:38-46, 1989).

Self-antigens are also continuously processed and presented as antigen fragments by the MHC glycoproteins to T cells (Jardetsky et al., Nature 353:326-329, 1991). Self recognition thus is intrinsic to the immune system. Under normal circumstances the immune system is tolerant to self-antigens and activation of the immune response by these self-antigens is avoided. When tolerance to self-antigens is lost, the immune system becomes activated against one or more self-antigens, resulting in the activation of autoreactive T cells and sometimes also the production of autoantibodies. This phenomenon is referred to as autoimmunity. As the immune response in general is destructive, i.e. meant to destroy the invasive foreign antigen, autoimmune responses can cause destruction of the body's own tissue.

It will thus be clear that fragments of the MIA protein will be expressed by the APC and that therefore also fragments of the MIA protein are capable of evoking an immune response. Also proteins of other species having a similar function or at least being structurally closely related to the human MIA protein might perform the same toleragenic effect. Thus, also homologous polypeptides or orthologs or parts thereof evoking the immune response are included in the invention.

Variations that can occur in a sequence, especially of smaller peptides, may be demonstrated by (an) amino acid difference(s) in the overall sequence or by deletions, substitutions, insertions, inversions or additions of (an) amino acid(s) in said sequence. Amino acid substitutions that are expected not to essentially alter biological and immunological activities have been described. Amino acid replacements between related amino acids or replacements which have occurred frequently in evolution are, inter alia Ser/Ala, Ser/Gly, Asp/Gly, Asp/Asn, Ile/al (see Dayhof, M. D., Atlas of protein sequence and structure, Nat. Biomed. Res. Found., Washington D.C., 1978, vol. 5, suppl. 3). Based on this information Lipman and Pearson developed a method for rapid and sensitive protein comparison (Science, 227:1435-1441, 1985) and determining the functional similarity between homologous polypeptides.

The protein according to the present invention includes the polypeptide comprising SEQ ID NO: 1 but also polypeptides with a similarity of at least 70%, preferably 90%, more preferably 95% are included. Also portions of such polypeptides still capable of conferring the toleragenic effects are included. Such portions may be functional per se, e.g. in solubilized form or they might be linked to other polypeptides, either by known biotechnological ways or by chemical synthesis, to obtain chimeric polypeptides.

As used herein the term similarity is as defined in NCBI-BLAST 2.0.10 [Aug-26-1999] (Altschul, Stephen F., Thomas L. Madden, Alejandro A. Schaffer, Jinghui Zhang, Zheng Zhang, Webb Miller, and David J. Lipman (1997) “Gapped BLAST and PSI-BLAST: a new generation of protein database search programs”, Nucleic Acids Res. 25:3389-3402). The program is used to search for sequence alignments using default settings. For amino acid alignments the BLOSUM62 matrix is used as a default and the similarity is indicated as the number of positives. No filtering of low compositional complexity is included.

The fragments of the MIA protein or homologous polypeptides are to be understood subsequences of the protein. “Subsequence” is understood to be defined as “a part” and should not be mistaken to encompass the entire protein. These subsequences have the following functional characteristics: i) peptides can be bound by the disease-associated MHC molecules, preferably HLA-DRB1*0101, DRB1*0401, DRB1*0404, DRB1*0408, DRB1*0405, DQB*0301, or DQB*0302, and ii) peptides must be able to provoke a T cell response in humans, preferably autoimmune patients, more preferably RA patients. Such a response can for example be measured in an in vitro T cell proliferation assay or in an assay for the detection of T cell cytokine production (e.g. ELISA or ELISPOT) (Coligan et al., Current Protocols in Immunology. John Wiley & Sons, Inc., 1998). Preferably the peptides must also be recognized by T cells in animals transgenic for the relevant human MHC class II molecules, as mentioned above, and human CD4 upon immunization with a MIA (poly)peptide.

The length of these subsequences is not important provided that it comprises the epitope to be recognised by the relevant MHC molecule. Preferably the subsequence has at least 9 consecutive amino acids of MIA. Preferably these peptides have an amino acid sequence of 9-55 amino acid residues. More preferably the peptides have an amino acid sequence of 9-35, in particular 9-25 amino acid residues. Much more preferred are peptides having an amino acid sequence of 9-15 amino acid residues. Highly preferred are peptides having an amino acid sequence of 13 or 14 amino acid residues. Most preferred are those peptides comprising SEQ ID NO: 11 or SEQ ID NO: 12.

Also within the scope of the invention are multimers of the peptides such as for example a dimer or trimer of the peptides according to the invention. A multimer according to the invention can either be a homomer, consisting of a multitude of the same peptide, or a heteromer consisting of different peptides.

It will be clear to those skilled in the art that the (poly)peptides may be extended at either side of the peptide or at both sides and still exert the same immunological function. The extended part may be an amino acid sequence similar to the natural sequence of the protein. However, the (poly)peptide might also be extended by non-natural sequences. Thus, MIA, as well as the fragments thereof having the anti-inflammatory function might be extended at either site with non-natural sequences. Therefore, e.g. polypeptides comprising SEQ ID NO:11 or SEQ ID NO:12 are part of the invention. The length of these peptides is preferably as indicated above. It will be clear that the (poly)peptide need not to exert its original function and as such might be inactive while still performing its immunological function according to the invention. The (poly)peptide according to the invention might be connected to MHC II molecules, such that the binding groove is occupied by the peptide. A flexible linker molecule, preferably also consisting of amino acid sequences might connect the peptide. The MHC molecules need not to possess their constant domains and might consist of their variable domains only, either directly connected to each other or connected through a flexible linker. The advantage of such a complex is that it might exist in a soluble, form and can directly be recognised by T cells.

The polypeptides according to the present invention therefore can be used in the preparation of a pharmaceutical to prevent inflammatory diseases.

The (poly)peptides, said (poly)peptides resembling the MHC Class II restricted T-cell epitopes present on the antigen comprising the MIA polypeptide or fragments thereof comprising these epitopes are very suitable for use in a therapy to induce systemic immune tolerance to said antigen in mammals, more specifically humans, suffering from T-cell mediated cartilage destruction, such as for example arthritis, more specifically rheumatoid arthritis.

More specifically the polypeptides can be used in the preparation of a pharmaceutical to induce specific T-cell tolerance in patients suffering from inflammatory diseases, preferably immune-cell mediated cartilage destruction. The immune cell, preferably is a T cell. The most preferred disease is arthritis, more preferably rheumatoid arthritis. In addition to a therapeutic treatment wherein patients suffering form an inflammatory disease are treated, MIA and the fragments thereof can also be used in a prophylactic treatment in patients which are susceptible to an inflammatory disease.

Treatment of autoimmune disorders with the peptides according to the invention makes use of the fact that systemic immune tolerance is induced to unrelated but co-localised antigens. The regulatory cells secrete in an antigen specific fashion pleiotropic proteins such as cytokines which may downmodulate the immune response.

Optionally such a treatment can be combined with the administration of other medicaments such as DMARDs (Disease Modifying Anti-Rheumatic Drugs e.g. sulfasalazine, anti-malarials (chloroquine, hydroxychloroquine) injectable or oral gold, methotrexate, D-penicillamine, azathioprine, cyclosporine, mycophenolate), NSAIDs (non steroidal anti inflammatory drugs), corticosteroids or other drugs known to influence the course of the disease in autoimmune patients.

The polypeptides according to the invention can also be used to modulate lymphocytes that are reactive to antigens other than said antigen but are present in the same tissue as the antigen i.e. proteins comprising the MIA polypeptide i.e. the polypeptide according to SEQ ID NO:1 or parts thereof. By the induction of antigen-specific T-cell tolerance, autoimmune disorders can be treated by systemic immune tolerance. More in general, the cells to be modulated are hematopoietic cells. In general, in order to function as a toleragen the peptide must fulfil at least two conditions i.e. it must possess an immune modulating capacity and it must be expressed locally usually as part of a larger protein.

The polypeptides according to the invention can be prepared by recombinant DNA techniques. A nucleic acid sequence coding for the protein, a peptide according to the invention, a multimer of said peptides or a chimeric peptide is inserted into an expression vector. Suitable expression vectors comprise the necessary control regions for replication and expression. The expression vector can be brought to expression in a host cell. Suitable host cells are, for instance, bacteria, yeast cells and mammalian cells. Such techniques are well known in the art, see for instance Sambrook et al., Molecular Cloning: a Laboratory Manual, Cold Spring Harbor laboratory Press, Cold Spring Harbor, 1989.

The (poly)peptides according to the invention can also be prepared by well known organic chemical methods for peptide synthesis such as, for example, solid-phase peptide synthesis described for instance in J. Amer. Chem. Soc. 85:2149 (1963) and Int. J. Peptide Protein Res. 35:161-214 (1990).

The (poly) peptides may be stabilised by C- and/or N-terminal modifications, which will decrease exopeptidase catalysed hydrolysis. The modifications may include: C-terminal acylation, (e.g. acetylation=Ac-peptide), N-terminal amide introduction, (e.g. peptide-NH₂) combinations of acylation and amide introduction (e.g. Ac-peptide-NH₂) and introduction of D-amino acids instead of L-amino acids (Powell et al., J. Pharm. Sci., 81:731-735, 1992).

Other modifications are focussed on the prevention of hydrolysis by endopeptidases. Examples of these modifications are: introduction of D-amino acids instead of L-amino acids, modified amino acids, cyclisation within the peptide, introduction of modified peptide bonds, e.g. reduced peptide bonds ψ[CH₂NH] and e.g. peptoids (N-alkylated glycine derivatives) (Adang et al., Recl. Trav. Chim. Pays-Bas, 113:63-78, 1994 and Simon et al., Proc. Natl. Acad. Sci. USA, 89:9367-9371, 1992).

The present invention provides a method to treat patients suffering from or susceptible to inflammatory autoimmune diseases, by administration of a pharmaceutical preparation comprising the (poly)peptide according to the invention. The (poly)peptide comprises T-cell epitopes, which are recognised by and are able to stimulate autoreactive T-cells. These T cells may be found e.g. in the blood of patients suffering from inflammatory disorders. Such patients may suffer from diseases like Graves' diseases, juvenile arthritis, primary glomerulonephritis, polyarthritis, osteoarthritis, Sjögren's syndrome, myasthenia gravis, rheumatoid arthritis, Addison's disease, primary biliary sclerosis, uveitis, systemic lupus erythematosis, inflammatory bowel disease, multiple sclerosis or diabetes.

Thus, according to the invention mammals suffering or susceptible to an inflammatory disease may be treated by administering a composition comprising MIA and/or fragments thereof that will have anti-inflammatory effects together with a pharmaceutically acceptable carrier. Preferably a systemic immune tolerance inducing amount of a composition comprising MIA and/or fragments thereof that will induce said systemic immune tolerance are administered. More preferably a T cell specific tolerance-inducing amount is administered. The inflammatory disease preferably is an immune cell mediated cartilage destruction disease, more preferably arthritis, even more preferably rheumatoid arthritis.

The forgoing compositions might also comprise peptides comprising a subsequence of MIA having at least 9 consecutive amino acids of MIA. Preferably the compositions comprise a peptide comprising SEQ ID NO:11 or SEQ ID NO:12. Even more preferably the peptide consists of the SEQ ID NOs 11 or 12. Thus, these peptides can be used as a therapeutic substance. These peptides therefore can also be used for the manufacture of a pharmaceutical preparation against inflammatory diseases as described in the foregoing.

Administration of the pharmaceutical composition according to the invention will induce systemic immune tolerance, in particular tolerance of the specific autoreactive T cells of these patients, to the autoantigenic proteins in the articular cartilage under attack and other self antigens which display the identified MHC Class II binding T cell epitopes characterised or mimicked by the amino acid sequences of one or more of the peptides according to the invention. The induced tolerance thus will lead to a reduction of the local inflammatory response in the articular cartilage under attack.

The (poly)peptides according to the invention have the advantage that they have a specific effect on the autoreactive T cells thus leaving the other components of the immune system intact as compared to the non-specific suppressive effect of immunosuppressive drugs.

Systemic immune tolerance can be attained by administering high or low doses of peptides according to the invention. The amount of peptide will depend on the route of administration, the time of administration, the age of the patient as well as general health conditions and diet.

In general, a dosage of 0.01 to 10000 μg of peptide per kg body weight, preferably 0.05 to 500 μg, more preferably 0.1 to 100 μg of peptide can be used.

Pharmaceutical acceptable carriers are well known to those skilled in the art and include, for example, sterile saline, lactose, sucrose, calcium phosphate, gelatin, dextrin, agar, pectin, peanut oil, olive oil, sesame oil and water. Other carriers may be, for example MHC class II molecules, if desired embedded in liposomes.

In addition the pharmaceutical composition according to the invention may comprise one or more adjuvants. Suitable adjuvants include, amongst others, aluminium hydroxide, aluminium phosphate, amphigen, tocophenols, monophosphenyl lipid A, muramyl dipeptide and saponins such as Quill A. Preferably, the adjuvants to be used in the tolerance therapy according to the invention are mucosal adjuvants such as the cholera toxin B-subunit or carbomers, which bind to the mucosal epithelium. The amount of adjuvant depends on the nature of the adjuvant itself.

Furthermore the pharmaceutical composition according to the invention may comprise one or more stabilisers such as, for example, carbohydrates including sorbitol, mannitol, starch, sucrosedextrin and glucose, proteins such as albumin or casein, and buffers like alkaline phosphates.

Suitable administration routes are e.g. intramuscular injections, subcutaneous injections, intravenous injections or intraperitoneal injections, oral administration and nasal administration such as sprays. Intranasal administration is preferred.

For testing the ability of the (poly)peptides to modulate (auto)immune responses several murine models have been shown to be suitable, such as collagen induced arthritis in mice (CIA), adjuvant arthritis in rats, experimental allergic encephalomyelitis in mice and non-obese diabetes in mice (NOD). Antigen may be administered intravenously, intraperitoneally, orally or nasally in such models (review by Liblau et al., Immunol. Today 18:599-603, 1997). To facilitate the read-out in these models, it is of importance to increase the confidence interval. According to the present invention it has been found that incidence and clinical score in arthritis models can be improved by combining the original trigger of arthritis, e.g. collagen type II in CIA with a peptide derived from the extracellular matrix protein aggrecan. This peptide might preferably be administered simultaneously with the original trigger although a separate administration might also be possible.

The following examples are illustrative for the invention and should in no way be interpreted as limiting the scope of the invention.

### LEGENDS TO THE FIGURES

FIG. 1 Schematic representation of the pNGVI-MIA(His₇) DNA construct. The MIA(His₇) coding sequence was cloned as an EcoRI-BamHI fragment behind the SV40 early promoter/enhancer/origin.

FIG. 2 Incidence of clinical signs of collagen type II induced arthritis in DBA/1 mice (n=15/group). Monitoring included all 4 paws per animal (for criteria, see legend of FIG. 3). The animals were treated at days 20, 25, and 30 following arthritis induction by intranasal administration of 30 μg MIA (▪) or saline (●). A two-tailed chi-square statistical test (in comparison with the saline-treated control group) indicated that P=0.020 (*).

FIG. 3 Progression of collagen type II induced arthritis after intranasal administration on days 20, 25 and 30 following arthritis induction of 30 μg MIA (▪) or saline (●) in DBA/1 mice. Monitoring included all 4 paws per animal and was according to criteria described by Joosten et al (1997, J. Immunol., 159:4094-4102): score 0.5, significant changes; score 1.0, moderate changes; score 1.5, marked changes; score 2.0, severe arthritis accompanied by maximal swelling, redness, and ankylosis. Data represent the mean score per group of mice (n=15). Severity of disease development is largely suppressed in MIA-treated animals. A two-tailed Mann Whitney statistical test with 95% confidence level (in comparison with the saline-treated control group) indicated that P<0.05 (*), i.e. P=0.0316 at day 28, P=0.0377 at day 30, and P=0.0268 at day 35. For day 33 P=0.0545 (**).

FIG. 4 Intranasal application of MIA protects against joint destruction in collagen type II arthritis. Radiography (X-ray imaging) was performed on hind paws of individual mice (n=15 DBA/1 mice/group) at the end of the experiment (day 35). Radiographs were scored with the use of a stereomicroscope under low magnification. A score of 0 to 5 was given to each paw according to the following guidelines: score 0: no changes; score 1: minor changes; score 2: moderate changes; score 3: marked changes; score 4: severe changes; score 5: complete destruction reflecting the severe arthritis that was also externally visible in a number of animals. Data represent the mean +/−SEM for the saline-treated mice (white bar) and the MIA-treated mice (black bar, 30 μg doses). Statistical comparison of data using a two-tailed Mann Whitney test with a 95% confidence level showed that P=0.0065 (*).

FIG. 5 Detection of MIA gene expression in RA cartilage. On cDNA derived from arthritic knee cartilage of 5 RA patients RT-PCR was performed with MIA-specific (lanes 2-6) or GAPDH-specific oligonucleotides (lanes 8-12). Per PCR reaction 5 ill were separated on agarose gel with the 100-bp-ladder (Gibco-BRL) as a fragment length marker (lane 7). The broad band in the middle of lane 7 represents the 600 bp marker fragment. RT-PCR control reactions without template cDNA using MIA and GAPDH specific oligonucleotides are shown in lanes 1 and 13, respectively.

FIG. 6 Incidence of clinical signs of collagen type II induced arthritis in DBA/1 mice (n=15/group). Monitoring included all 4 paws per animal (for criteria, see legend of FIG. 7). The animals were treated at days 20, 25, and 30 following arthritis induction by intranasal administration of 10 μg MIA peptide (▴), 30 μg MIA peptide (▪) or saline (●). A two-tailed Fisher statistical test (in comparison with the saline-treated control group) indicated P<0.05 (*), i.e. P=0.042 at day 30 and P=0.014 at day 33. A two-tailed chi-square statistical test indicated P=0.040 at day 37.

FIG. 7 Progression of collagen type II induced arthritis after intranasal administration on days 20, 25 and 30 following arthritis induction of 10 μg MIA peptide (▴), 30 μg MIA peptide (▪) or saline (●). Monitoring included all 4 paws per animal and was according to criteria described by Joosten et al (1997, J. Immunol., 159:4094-4102): score 0.5, significant changes; score 1.0, moderate changes; score 1.5, marked changes; score 2.0, severe arthritis accompanied by maximal swelling, redness, and ankylosis. Data represent the mean score per group of mice (n=15 DBA/1 mice/group). Severity of disease development is largely suppressed in 10 μg MIA peptide-treated animals. A two-tailed Mann Whitney statistical test with 95% confidence level (in comparison with the saline-treated control group) showed that P=0.0388 at day 33 (*), and P-0.0574 at day 37 (**). In a one-tailed test P=0.0287 at day 37 (**).

FIG. 8 Individual arthritis scores in a collagen type II induced arthritis after intranasal administration on day 20, 25 and 30 following arthritis induction of 10 μg MIA peptide (▴), 30 μg MIA peptide (▪) or saline (●) in DBA/1 mice. Monitoring included all 4 paws per animal (for criteria, see legend of FIG. 7). For each treatment group the median is indicated of the individual arthritis score of each mouse at the end of the experiment on day 37. Statistical analyses using a Mann Whitney test with 95% confidence level (in comparison with the saline-treated control group) showed that for the group treated with 10 μg MIA/dose P=0.0574 in a two-tailed test and P=0.0287 in a one-tailed test (**).

FIG. 9 Intranasal application of MIA peptide protects against joint destruction in collagen type II arthritis. Radiography (X-ray imaging) was performed on hind paws of individual mice (n=15 DBA/1 mice/group) at the end of the experiment (day 37). Radiographs were scored with the use of a stereomicroscope under low magnification. A score of 0 to 5 was given to each paw according to the following guidelines: score 0: no changes; score 1: minor changes; score 2: moderate changes; score 3: marked changes; score 4: severe changes; score 5: complete destruction reflecting the severe arthritis that was also externally visible in a number of animals. Data represent the mean +/−SEM for the saline-treated mice (white bar) and the MIA-treated mice (black bar, 10 fig doses and arched bar, 30 μg doses). Statistical comparison of data using a two-tailed Mann Whitney test with a 95% confidence level showed that P=0.0010 (*).

FIG. 10 DTH responses at 24 hours (black bars) and 48 hours (white bars) after challenge in DBA/1 mice. Mice were immunised and challenged with MIA (2 sets of bars on the right) or, as a positive control on DTH induction, with ovalbumin (2 sets of bars on the left). At 5, 10 and 15 days before immunisation mice were treated by intranasal administration with 30 μg MIA, 30 μg ovalbumin (positive treatment control), or saline (negative treatment control). Data represent the mean antigen-specific paw swelling +/−SEM. Statistical comparison of the 24 h data of the protein-treated groups (MIA or ovalburnin, intranasally) with the corresponding saline-treated controls using a one-tailed Mann Whitney test with 95% confidence level, showed that P<0.05 (*).

FIG. 11 DTH response 24 hours (black bars) and 48 hours (white bars) after challenge in Balb/c fi mice. Mice were immunised and challenged with MIA (2 sets of bars on the right) or, as a positive control on DTH induction, with ovalbumin (2 sets of bars on the left). At 5, 10 and 15 days before immunisation mice were treated by intranasal administration with 30 μg MIA, 30 μg ovalbumin (positive treatment control), or saline (negative treatment control). Data represent the mean antigen-specific paw swelling +/−SEM. Statistical comparison of the protein-treated groups (MIA or ovalbumin, intranasally) with the corresponding saline-treated controls using a one-tailed Mann Whitney test with 95% confidence level, showed that P<0.05 (*).

FIG. 12 T cell proliferation with human lymphocytes of RA patients (white bars, donors 1-5) and healthy donors (black bars, donors 6-10). Cells were cultured with 0.2 μg plate-bound anti-CD3 antibodies. After 3 days 0.1 μCi ³H-thymidine was added and cells were incubated for 18 hours. Incorporation of ³H-thymidine, as a measure for cell proliferation, was determined by gas scintillation. Data represent the mean stimulation index.

FIG. 13 T cell proliferation with human lymphocytes of RA patients (white bars, donors 1-5) and healthy donors (black bars, donors 6-10). Cells were cultured with 0.5 μg MIA. After 6 days 0.1 μCi ³H-thymidine was added and cells were incubated for 18 hours. Incorporation of ³H-thymidine, as a measure for cell proliferation, was determined by gas scintillation. Data represent the mean stimulation index.

### EXAMPLES

### Example 1

DNA Cloning and Production/Purification of Recombinant MIA(his7)

cDNA Cloning

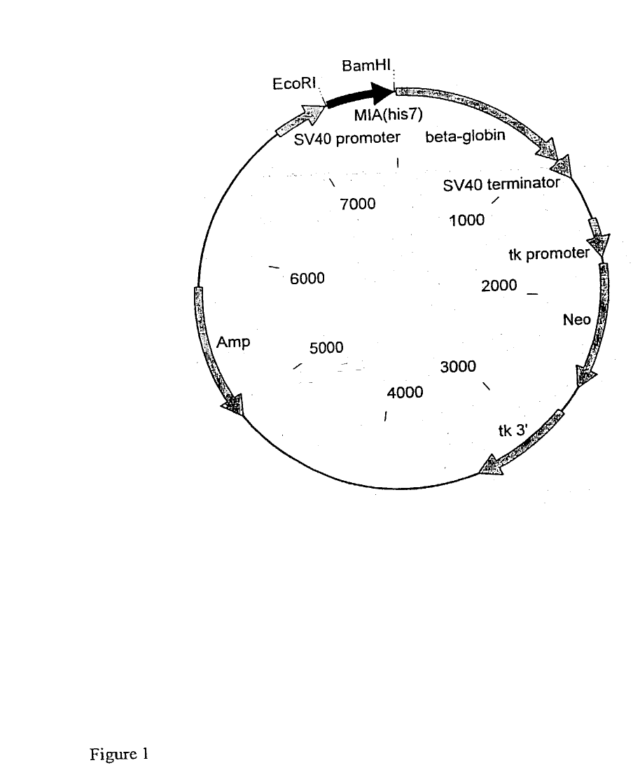

Standard culturing was performed with human melanoma cells in DMEM/Hamm's F12 (1:1) medium in the presence of 10% fetal calf serum. Prior to RNA isolation, cultured monolayer cells were washed once with ice cold PBS buffer. RNA was isolated with RNAzol B (Campro Scientific). First strand cDNA synthesis was performed with Superscript II (Gibco-BRL) and random 6-mer primers on ˜4 μg of total RNA. For MIA cDNA cloning via RT-PCR on cDNA from GMM3 cells, two oligonucleotides were designed: sense primer (5′ ATATGAATTCGCCACCATGGCC CGGTCCCTGGTGTGCCTT 3′) (SEQ ID NO:4) and antisense primer (5′ ATATGGATCCTTTAATGGTGATGGTGATGGTGATGGCAGTAGAAATCCCAT TTGTC 3′) (SEQ ID NO:5). The sense primer contained an optimised translational start region according to Kozak (1999, Gene 234:187-208), and the antisense primer an optimised translational stop-region (McCaughan et al., 1995, Proc. Natl. Acad. Sci. USA 92:5431-5435); italics) and 7 His codons following the Cys-130 codon of MIA (Blesch et al., 1994, Cancer Res. 54:5695-5701). PCR was performed in a Perkin Elmer 9600: 1 cycle 5 min 94° C., 35 cycles 30 sec 94° C./30 sec 55° C./1 min 72° C., 1 cycle 5 min 72° C. with 400 ng/primer, 200 μM dNTPs, and 1 u Taq polymerase in 100 μl total volume. PCR amplification products were isolated from agarose gel and cloned into vector pCR2.1 (Invitrogen). The cDNA insert of MIA-pCR2.1 clone 2 was sequenced in two directions (SEQ ID NO:2). For cDNA subcloning into the eukaryotic expression vector pNGVI (EMBL accession number X99274), the MIA cDNA was digested from MIA-pCR2.1 clone 2 with restriction enzymes EcoRI and BamHI and ligated into pNGV1 behind the SV40 early promoter, resulting in pNGV1-MIA(His7)-clone1 (FIG. 1). The plasmid encodes a protein with the sequence of SEQ ID NO:1 wherein the last amino acid Q is replaced by (His)₇.

Transfection of CHO Cells With pNGV1-MIA(His7) DNA

CHO cells (ATCC CCL61) were cultured in DMEMJHamm's F12 containing 5% FCS (Harlan sera lab). The PNGV1-MIA(His7) construct was transfected to CHO-K1 using Transfectam (Promega) and selection medium DMEM/Hainu's F12 containing 5% FCS and 0.8 mg/ml neomycin (G418 sulphate Gibco BRL Life technology, filter sterilised using a 0.22 μM Millipore SLGV025BS filter). The transfected cells were frozen in DMEM F12, 10% FCS, 10% DMSO as a pool of cells at −140° C. These stocks were thawed and cultured in T25 roux flask in DMEM F12, 5% FCS plus 0.8 mg/ml neomycin. After three days single cell cloning was carried out by plating 20, 10 and 5 cells/well in 96 wells plates. Clones were selected by visual inspection. Two weeks after cloning the clones were transferred to 6 well plates and grown to 90% confluence. Next, cells were cultured o/n in serum-free medium (containing 0.8 mg/ml neomycin) and expression was allowed to continue for one day. MIA-His7 expression was detected using a 96 wells dotblot (see below). The highest producing transfectants were scaled up and frozen in ampoules at −140° C. Serum-free culture supernatant was also analysed on SDS-PAGE followed by Western blotting and subsequent detection with anti-His6 monoclonal antibody (Dianova GMBH, cat. no. Dia 900 lot. no. 100696, diluted 1000 times). The blocking and antibody incubations were carried as described for the dotblot procedure.

Detection of MIA-His7 Using a Dotblot.

Samples taken from conditioned media of the CHO.pNGV1.MIA(His7) cloning were spotted on a nitrocellulose filter (Biorad 0.45 μM lotno 9473) using a vacuum dotblot apparatus (Hybri.DOT, BRL, The Netherlands). The dotblot was incubated for 30 min with Amersham Life science liquid block buffer (diluted 10 times in ECF buffer; 0.1 M Tris-HCl pH 7.5, 0.3 M NaCl). Next, the dotblot was incubated with 0.2 μg/ml mouse-anti(His6)-tag (Dianova GMBH cat. no. Dia 900 lot. no. 100696) diluted in PBST at RT. After three times washing with PBST for five minutes at RT, the dotblot was incubated with 250 ng/ml anti-mouse-IgG-HRP (Promega catno 3624512) for two hours at RT. After three times washing with PBST for five minutes at RT, detection was performed using ECL (Amersham life science batch 96) according to the manufacturers instructions.

The dotblot detection indicated that the transfectants 18, 32 and 37 had the highest expression per 2.104 cells/ml. Therefore, these clones were selected for further analysis. Pilot stability studies in spinners were carried out.

Stability Studies of MIA-His7 Transfectants in Spinner Cultures

Cells of transfectants 18, 32, and 37 were cultured to 100% confluence in T175 Roux flask. Spinners containing 250 ml DMEM F12 modified, 5% FCS and 250 mg carriers (cultisphereS, P Biolytica AB, Art. DG-2001-ZZ) were seeded in duplo. FCS was reduced stepwise and finally replaced with DMEM F12+0.5 μg/l insulin and 5 mg/l transferrin. During at least four weeks the last mentioned medium was replaced every 2 or 3 days and expression of MIA-His7 was analysed by Western blot. Supernatants of all spinners were collected and stored at −20° C. for later use. The stability of these clones were tested during 34-39 days. Every time the medium was refreshed samples were taken, concentrated 5 times using Amicon micro concentrators (cut-off 10 kDa, no. 424070) and tested for MIA(His7) using the Western blot procedure. After 34-39 days all six spinners were still expressing the MIA-His7 protein. On Western blot the bands of the last samples bad the same intensity as the first samples. Thus, according to this analysis, the transfectants were stable with respect to production of MIA(His7).

The CHO-K1-pNGV1.MIA(His7) clone 18 was selected for production in a 5 L fermenter. The harvest medium of the fermenter contained DMEM F12+0.5 μg/l insulin and 5 mg/l transferrin. The harvest medium from the fermenter was filtered stepwise from 3 μm to 0.8 μm to 0.22 μm and collected in plastic bags at 4° C.

Purification of the MIA(His7) Protein from CHO-K1 Conditioned Medium

About 12 l conditioned medium from fermenter cultures, buffered with 20 mM sodium phosphate pH 7.0 was loaded onto a SP-Sepharose Streamline XL (Pharmacia Biotech codeno 17-5076-01) column (XK50, 300 ml) at a flow rate of 12 ml/min (Pharmacia Biotech piston pump P900). This process was monitored using a UV-detector (Monitor UV-900 Pharmacia biotech). A single wash step with 20 mM sodium phosphate 0.10 M NaCl pH 7.0 was carried out. The MIA(His7) that was bound to the column was eluted with 20 mM sodium phosphate 0.40 M NaCl, pH 7.0 and collected in 50 ml fractions (Pharmacia biotech frac-900). The fractions were analysed by SDS-PAGE and Western blotting. The fractions containing MIA-His7 were pooled and subjected to a gelfiltration column. After equilibration of the gel filtration column (XK 26/70 300 ml Superdex 75, Pharmacia Biotech codeno 17-1044-01) with 20 mM sodium phosphate, 0.4 M NaCl, pH 7.0, the SP-Sepharose pool was applied onto the column in portions of 6 ml. The proteins were eluted with a flow of 2.0 ml/min. The fractions were analysed by SDS-PAGE and Western blotting, the fractions containing the MIA(His7) protein were pooled. This was confirmed by Western blot. To determine the purity of the pooled fractions using SDS-PAGE (16×20 cm), 20 μg of purified protein was loaded. The protein concentration was determined using the Pierce BCA protein assay reagent kit. The gel was scanned using a densitometer (GS-700, Bio-rad) and the scan was analysed using the molecular analyst software (Bio-rad). From the scanning data it was concluded that the MIA(His7) preparation was over 92% pure. The identity of the purified MIA(His₇) protein was positively confirmed by MALDI and ESI mass determination, followed by N-terminal amino acid sequencing.

### Example 2

Intranasal Tolerance Induction With MIA Ameliorates Clinical and Radiological Signs of Collagen Type II Induced Arthritis in DBA-1 Mice

In order to investigate the immunomodulatory potential of MIA on arthritis disease development, MIA (prepared as in example 1) was intranasally administered to DBA/1 mice during early phases of arthritis development. Male DBA/1 mice were obtained from Bomholtgaard (Ry, Denmark). Mice were immunised (day 0) with 30 μg aggrecan peptide (aa: AGWLADRSVRYPI, SEQ ID NO:6) and 100 μg bovine collagen type II in Mycobacterium tuberculosis-enriched (2 mg/ml final concentration) complete Freunds adjuvant. On day 21, mice received an intraperitoneal booster injection with 30 μg aggrecan peptide and 100 μg bovine collagen type II in saline. On days 20, 25 and 30 mice were treated via intranasal administration with either MIA (30 μg/animal/dose, n=15) or, as a control, with buffer (15 μl saline) alone (n—15). Disease incidence and progression of artritis activity was followed visually over time, starting at day 0 with 2-3 days intervals (during treatment, observation at days 21, 23, 26, 28, 30, 33 and 35 following arthritis induction). Clinical severity of arthritis was graded on a scale of 0 to 2 per paw.

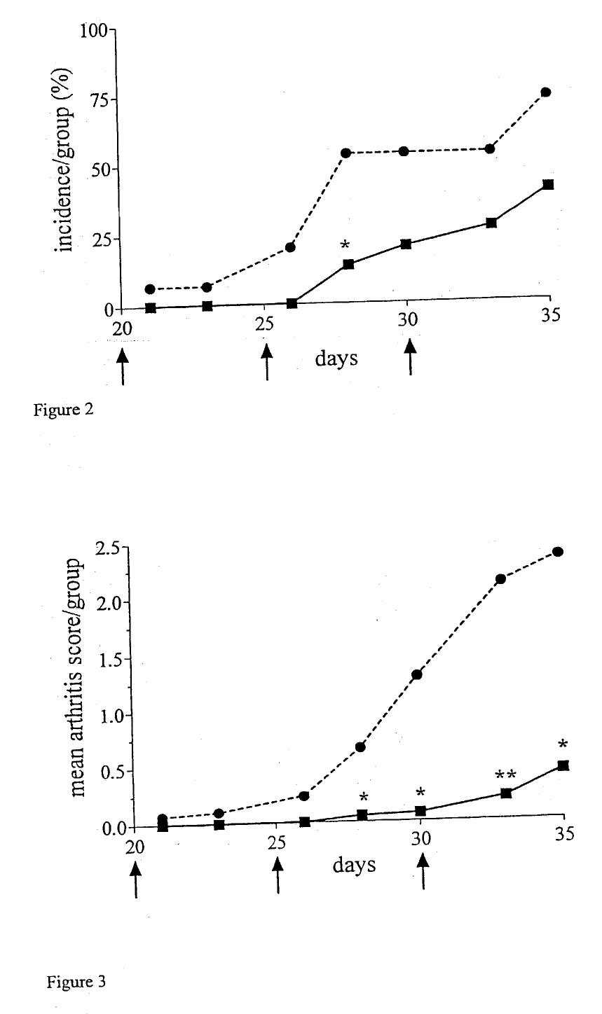

Starting at day 21 arthritis gradually developed. At day 35 73% of the saline-treated animals showed clinical signs of arthritis (FIG. 2). In contrast, in the MIA-treated group only 40% of the animals developed clinical signs of disease (FIG. 2). Furthermore, it was noted that clinical signs of arthritis (day 21-day35) were less severe in the MIA-treated group than in the saline-treated mice (FIG. 3), indicating that intranasal administration of MIA reduces the local inflammation process associated with arthritis development.

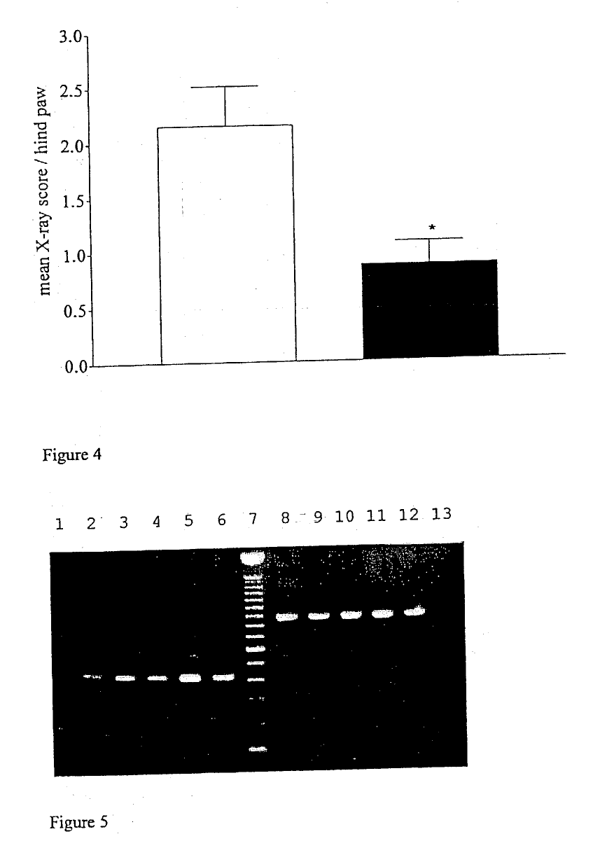

To examine whether MIA can protect against joint destruction, radiography (X-ray imaging) was performed on hind paws of individual mice at the end of the experiment (day 35). Radiographs were scored with the use of a stereomicroscope under low magnification. Destructive processes were scaled on from 0 (no changes) to 5 (complete destruction of a joint and/or new bone formations). Intranasal administration of MIA significantly inhibited joint destruction as compared to the mice treated with saline alone (FIG. 4).

### Example 3

Detection of MIA Gene Expression in Arthritic Cartilage

Expression of the MIA gene in diseased tissue was detected via RT-PCR with MIA-specific oligonucleotides (SEQ ID NO:7 and SEQ ID NO:8) on cDNA that was derived from cartilage samples of 5 RA patients. The arthritic cartilage was obtained during joint replacement surgery of the knee. Chondrocytes were isolated enzymatically from the cartilage (Cornelissen et al., 1993, J. Tiss. Cult. Meth. 15:139-146) upon which RNA was isolated using Trizol (Gibco-BRL) or RNAzol B (Campro Scientific). With 1 μg of total RNA the synthesis of cDNA was performed using Superscript II (Gibco-BRL) in a total volume of 20 μl. For RT-PCR on MIA and on housekeeping gene GAPDH, as positive control, 0.5 μl cDNA per reaction was used. PCR was performed in a Perkin Elmer 9600: 1 cycle 5 min 94° C., 35 cycles 30 sec 94° C./30 sec 55° C./1 mm 0.72° C., 1 cycle 5 min 72° C. with 50 ng/primer, 200 μM dNTPs, and 2.5 u Taq polymerase (Pharmacia) in 25 ill total volume. Oligonucleotides specific for GAPDH were SEQ ID NO:9 and SEQ ID NO:10. PCR samples were analysed on agarose gel (FIG. 5). Lanes 2-6 show clear signals of MIA cDNA amplification product of the expected length for all 5 arthritis patients, while GAPDH amplification signals are in the same order of magnitude for each cDNA preparation (lanes 8-12). The RT-PCR data indicate that the MIA gene is expressed in diseased tissue, i.e. afflicted knee cartilage, of 5/5 RA patients tested. It is likely that the MIA gene indeed is expressed in diseased articular cartilage of at least a considerable percentage of RA patients. Consequently, it is to be expected that the MIA protein is synthesised in diseased cartilage of RA patients.

### Example 4

Intranasal Tolerance Induction With MIA-Derived Peptide Ameliorates Clinical and Radiological Signs of Collagen Type II Induced Arthritis in DBA/1 Mice

In order to investigate the immunomodulatory potential of a MIA-derived fragment on arthritis disease development, a peptide was selected from the MIA amino acid sequence (SEQ ID NO:1). The selection was based on a consensus sequence motif that predicts the binding of a corresponding peptide to RA-relevant MHC class II DR molecules. As a result the MIA sequence of amino acids 100-108 was identified as a predicted DR-binding peptide (SEQ ID NO:11). Flanked by 2 additional amino acids on either side, 13-mer MIA peptide 98-110 (amino acids: ARLGYFPSSIVRE; SEQ ID NO:12) was synthesised by Neosystem (Strasbourg, France) and delivered as a more than 95% pure preparation. The MIA peptide (SEQ ID NO:12) was intranasally administered to DBA/1 mice during early phases of induced arthritis development. Male DBA/1 mice were obtained from Bomholtgaard (Ry, Denmark). Mice were immunised (day 0) with 30 μg aggrecan peptide (amino acids: AGWLADRSVRYPI, SEQ ID NO:6) and 100 μg bovine collagen type II in Mycobacterium tuberculosis-enriched (2 mg/ml final concentration) complete Freunds adjuvant. On day 21, mice received an intraperitoneal booster injection with 30 μg aggrecan peptide and 100 μg bovine collagen type II in saline. On days 20, 25 and 30 mice were treated via intranasal administration with either MIA peptide (10 and 30 μg/animal/dose, n=15/group) or, as a control, with buffer alone (15 μl saline, n=15/group). Disease incidence and progression of arthritis activity was followed visually over time, starting at day 0 with 2-3 days intervals (during treatment, observations at days 21, 23, 26, 28, 30, 33, 35 and 37 following arthritis induction). Clinical severity of arthritis was graded on a scale of 0 to 2 per paw. The experiment was performed as a double-blinded study, randomized in three blocks (5 animals per cage).

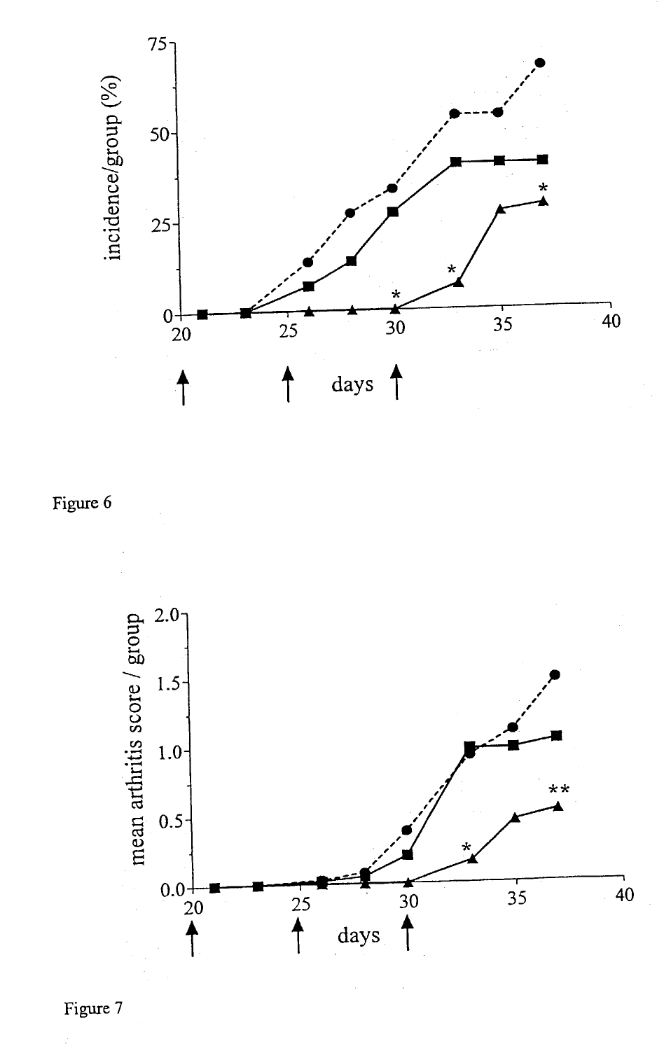

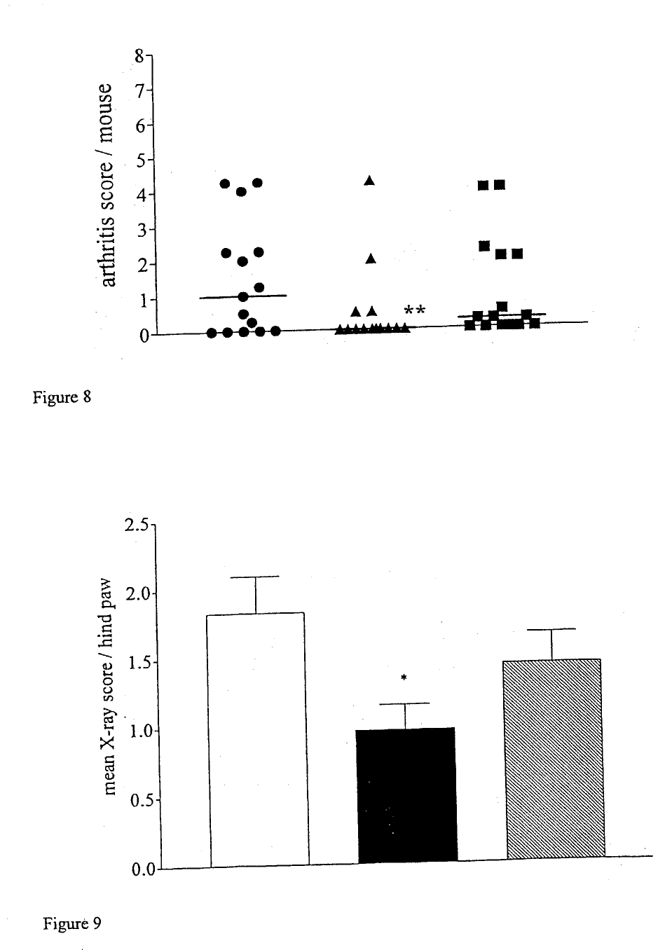

Starting at day 21 arthritis gradually developed. At day 37 67% of the saline-treated animals showed clinical signs of arthritis (FIG. 6). In contrast, in the MIA peptide-treated group (10 μg/animal/dose) only 28% of the animals developed clinical signs of disease (FIG. 6). Furthermore, it was noted that clinical signs of arthritis (day 21-day 37) were significantly less severe in the MIA peptide-treated group (10 μg/animal/dose) than in the saline-treated mice (FIG. 7) indicating that intranasal administration of MIA peptide reduced the local inflammation process associated with arthritis development. Although treatment with 30 μg/animal/dose of MIA peptide yielded less amelioration as compared to 10 μg/animal/dose, still, arthritis scores and incidence values generally were below those found in the saline-treated control mice (FIGS. 6 and 7). Amelioration on arthritis scores and incidence as a result of treatment with the MIA peptide (SEQ ID NO: 12) can also be seen per individual animal as shown in FIG. 8.

To examine whether the MIA peptide can protect against joint destruction, radiography (X-ray imaging) was performed on hind paws of individual mice at the end of the experiment (day 37). Radiographs were scored (double-blinded, randomized) with the use of a stereomicroscope under low magnification. Destructive processes were graded on a scale of 0 (no changes) to 5 (complete destruction of a joint and/or new bone formations) per paw. The results (FIG. 9) show that intranasal administration of MIA peptide significantly inhibited joint destruction as compared to the mice treated with saline alone.

### Example 5

Decreased Delayed Type Hypersensitivity Reaction (DTH) After Intranasal Administration With MIA.

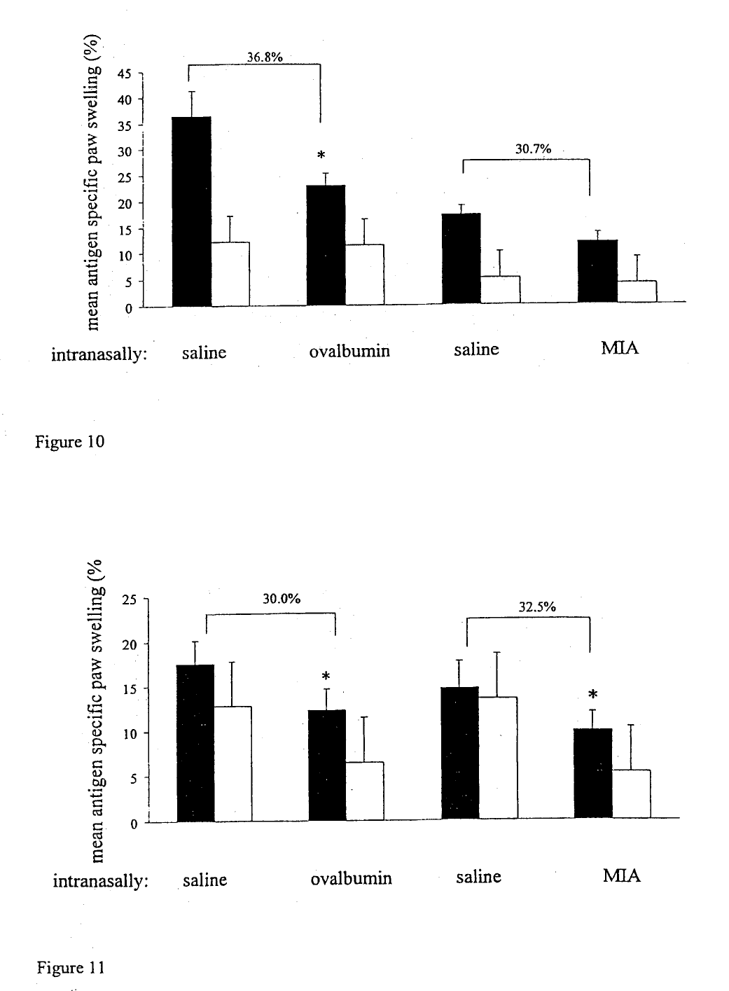

In order to show that it is possible to induce a regulatory T cell response that causes systemic immune tolerance by intranasal administration of MIA protein, a typical DTH test was performed. Mice were subcutaneously immunised with 10 fig MIA protein (for protein preparation see Example 1) in 50% Incomplete Freund Adjuvant at day 0. After 7 days all mice were challenged in the left footpad with 10 μg MIA protein in alum (1 mg/ml final concentration). The right footpad was injected with alum as a control. The typical DTI response (footpad swelling), resulting from T cell reactivity, was measured at 24 and 48 h after the challenge. To investigate the immune modulating role of MIA protein, the mice were treated by intranasal administration with 30 μg MIA or, as a control, with saline at 5, 10 and 15 days before immunisation (n−10 mice/group). As a positive control of the in vivo DTH model, mice were immunised (50 μg/mouse) and challenged (10 μg/mouse) with ovalbumin and treated intranasally (50 μg/mouse) with ovalbumin or saline. Ovalbumin has been described as being able to induce a regulatory T cell response in a DTH test. DTH responses were measured in both male DBA/1 mice (derived from Bomholtgaard) and in female Balb/c mice (derived from Charles River) and are shown in FIGS. 10 and 11, respectively. As expected in a DTH reaction, footpad swelling at 48 h after the challenge was always decreased as compared to 24 h. From FIGS. 10 and 11 it can be concluded that in both mouse strains the DTH responses against MIA protein were decreased by about 30% as a result of the intranasal administration of MIA protein. With ovalbumin as a positive control similar reductions were observed. These data are in agreement with the induction of an immune regulatory T cell population by intranasal administration of MIA protein leading to systemic immune tolerance.

### Example 6

Detection of T Cell Responses Against MIA Protein With Human Lymphocytes of RA Patients and Healthy Donors

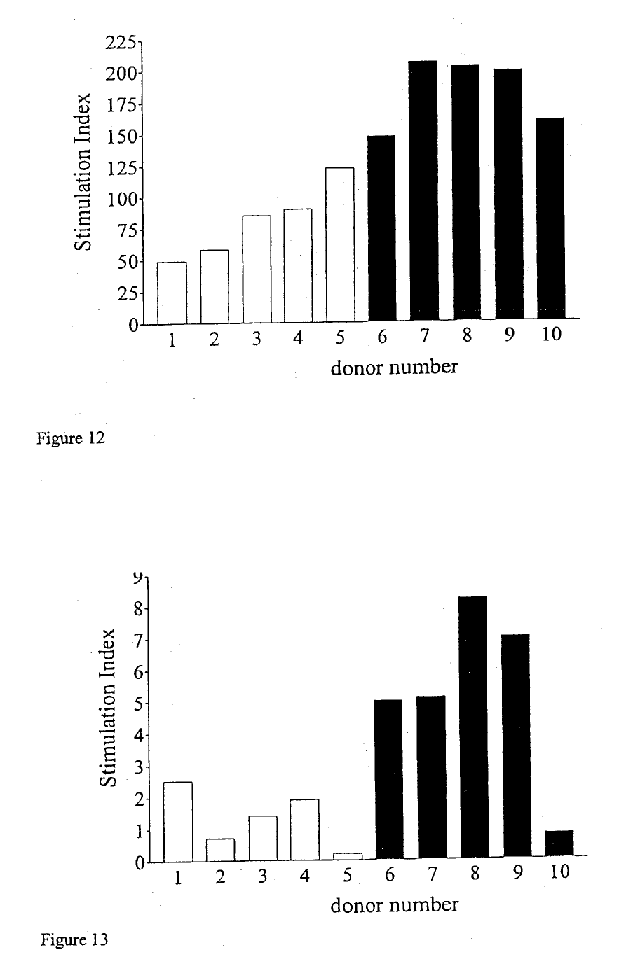

In order to show whether the MIA protein is recognized by T cells, either from healthy donors or from RA patients, a T cell proliferation experiment was performed. Human lymphocytes were isolated from heparinised venous peripheral blood of 5 healthy donors and 5 RA patients by standard centrifugation on Ficoll-Paque and were gradually frozen in 10% DMSO to −140° C. (Kryo 10 Series). All patients were diagnosed as RA according to the revised criteria formulated by the American Rheumatology Association (Arnett et al, 1988, Arthritis & Rheumatism 31:315-324) and found rheumatoid factor positive. Cells were thawed and resuspended gradually in culture medium (DMEM F12 containing 50% heat inactivated foetal calf serum (FCS)). Cells were plated in culture medium in flat-bottom 96-wells plates (Nunc) in a volume of 100 ill (1.5×10⁵ cells/well, 10% FCS final concentration). The cells were cultured for 6 days with 0.5 μg MIA protein (for protein preparation see Example 1) at 37° C. and 5% CO₂ in humidified air. Also cells were cultured in the presence or absence of anti-CD3 antibodies coated to the plate (CLB, The Netherlands, clone T3/2 16A9, 1 μg/ml, 200 μl/well, plates were coated for 18 h and stored at room temperature). The T cell response on an anti-CD3 challenge is regarded as a measure of the overall reactivity a T cell population is able of. Each in vitro stimulation was performed and measured in 5 separate wells. Of the supernatant 50 ill were removed from the wells after 3 days for the anti-CD3 stimulation and after 6 days for the MIA stimulation. Subsequently, 25 Ill culture medium containing 0.1 μCi ³H-thymidine was added to each well followed by 18 h incubation. Cells were harvested on glassfibre filters using a cell harvester. Incorporation of ³H-thymidine, as a measure for proliferation, was determined for 5 min by gas scintillation (Matrix 9600, Packard Canberra). Stimulation indices (SI) were calculated by dividing the anti-CD3-induced and antigen-induced signals by their background signals and are shown in FIGS. 12 and 13, respectively. The data from FIG. 12 show that upon a challenge with anti-CD3 the T cells of healthy donors respond roughly 2-3 fold stronger than T cells isolated from RA donors. From the stimulation indices of FIG. 13, T cells from 4/5 healthy donors apparently seem to recognise fragments of the MIA protein that are processed and presented by the T cells themselves. Responses above background level were detected for 3/5 RA patients although the level of responsiveness against MIA was low with the T cells from RA patients. This lower response level seems to correspond with the lower overall response potential of RA T cells as determined with the anti-CD3 challenge.

These data suggest that the potential of human peripheral T cells to respond to MIA is not associated with RA pathology per se. Since the ability of human T cells to respond to MIA seems not rare, it is likely that administration of tolerance-inducing amounts of MIA or fragments thereof, indeed would provoke a regulatory T cell response in humans.

1. Use of MIA and/or fragments thereof that will have anti-inflammatory effects for the manufacture of a pharmaceutical preparation against inflammatory diseases.

2. Use of MIA and/or fragments thereof that will induce systemic immune tolerance to the MIA antigen for the manufacture of a pharmaceutical preparation for the induction of said systemic immune tolerance in patients suffering from or susceptible to an inflammatory disease.

3. Use of MIA and/or fragments thereof that will induce specific T-cell tolerance to the MIA antigen for the manufacture of a pharmaceutical preparation for the induction of said specific T-cell tolerance in patients suffering from or susceptible to an inflammatory disease.

4. Use of claims 1-3 wherein the inflammatory disease is an immune-cell mediated cartilage destruction disease.

5. Use of claims 5 wherein the immune cell mediated cartilage destruction disease is arthritis, more specifically rheumatoid arthritis.

6. The use of claim 1-5 wherein said composition is administered by injection, orally or intranasally.

7. A method for treating mammals suffering from or susceptible to an inflammatory disease the method comprising administering a composition comprising MIA and/or fragments thereof that will have anti-inflammatory effects together with a pharmaceutically acceptable carrier.

8. A method for treating mammals suffering from or susceptible to an inflammatory disease the method comprising administering a systemic immune tolerance inducing amount of a composition comprising MIA and/or fragments thereof that will induce said systemic immune tolerance together with a pharmaceutically acceptable carrier.

9. A method for treating mammals suffering from or susceptible to an inflammatory disease the method comprising administering a T-cell specific tolerance inducing amount of a composition comprising MIA and/or fragments thereof that will induce said T-cell specific tolerance together with a pharmaceutically acceptable carrier.

10. The method of claims 7-9 wherein said inflammatory disease is an immune cell mediated cartilage destruction disease.

11. The method according to claim 10 wherein said disease is arthritis, more specifically rheumatoid arthritis.

12. The method of claim 7-11 wherein said composition is administered by injection, orally or intranasally.

13. A peptide comprising a subsequence of MIA having at least 9 consecutive amino acids of MIA.

14. A peptide of MIA having at least 9 amino acids, and comprising SEQ ID NO:11 or SEQ ID NO:12.

15. The peptide of claim 13 having the amino acid sequence of SEQ ID NO: 11 or SEQ ID NO:12.

16. A pharmaceutical composition comprising an effective amount of the peptide according to claims 13 or 14 and a pharmaceutically acceptable carrier.

17. Use of the peptides according to claims 13 or 14 for use as a therapeutic substance.


SEQUENCE LISTING (12 sequences)

SEQ ID NO: 1 - protein, 131 aa, Homo sapiens
MARSLVCLGVIILLSAFSGPGVRGGPMPKLADRKLCADQECSHPISMAVALQDYMAPDCRFLTIHRGQVVYVFSKLKGRGRLFWGGSVQGDYYGDLAARLGYFPSSIVREDQTLKPGKVDVKTDKWDFYCQ

SEQ ID NO: 2 - DNA, 433 bp, Homo sapiens
  CDS (13)..(426)
gaattcgccaccatggcccggtccctggtgtgccttggtgtcatcatcttgctgtctgccttctccggacctggtgtcaggggtggtcctatgcccaagctggctgaccggaagctgtgtgcggaccaggagtgcagccaccctatctccatggctgtggcccttcaggactacatggcccccgactgccgattcctgaccattcaccggggccaagtggtgtatgtcttctccaagctgaagggccgtgggcggctcttctggggaggcagcgttcagggagattactatggagatctggctgctcgcctgggctatttccccagtagcattgtccgagaggaccagaccctgaaacctggcaaagtcgatgtgaagacagacaaatgggatttctactgccatcaccatcaccatcaccattaaaggatcc

SEQ ID NO: 3 - protein, 137 aa, Homo sapiens
MARSLVCLGVIILLSAFSGPGVRGGPMPKLADRKLCADQECSHPISMAVALQDYMAPDCRFLTIHRGQVVYVFSKLKGRGRLFWGGSVQGDYYGDLAARLGYFPSSIVREDQTLKPGKVDVKTDKWDFYCHHHHHHH

SEQ ID NO: 4 - DNA, 40 bp, Homo sapiens
atatgaattcgccaccatggcccggtccctggtgtgcctt

SEQ ID NO: 5 - DNA, 56 bp, Homo sapiens
atatggatcctttaatggtgatggtgatggtgatggcagtagaaatcccatttgtc

SEQ ID NO: 6 - protein, 13 aa, Homo sapiens
AGWLADRSVRYPI

SEQ ID NO: 7 - DNA, 24 bp, Homo sapiens
attcgccaccatggcccggtccct

SEQ ID NO: 8 - DNA, 24 bp, Homo sapiens
ggtgatggtgatggcagtagaaat

SEQ ID NO: 9 - DNA, 25 bp, Homo sapiens
cccttcattgacctcaactacatgg

SEQ ID NO: 10 - DNA, 25 bp, Homo sapiens
ggtccaccaccctgttgctgtagcc

SEQ ID NO: 11 - protein, 9 aa, Artificial Sequence
  Description of Artificial Sequence  MIA amino acids 100-108
LGYFPSSIV

SEQ ID NO: 12 - protein, 13 aa, Artificial Sequence
  Description of Artificial Sequence  MIA amino acids 98-110
ARLGYFPSSIVRE